(19) United States
(12) Patent Application Publication
(10) Pub. No.: US 2021/0106440 A1
(43) Pub. Date: Apr. 15, 2021

(54) PASSIVE PROSTHETIC KNEE

(71) Applicants: Zeynolabedin Soleymani, Tehran (IR); Ayoob Davoodi, Izeh (IR); Rezvan Nasiri, Langarud (IR); Majid Nili Ahmadabadi, Tehran (IR)

(72) Inventors: Zeynolabedin Soleymani, Tehran (IR); Ayoob Davoodi, Izeh (IR); Rezvan Nasiri, Langarud (IR); Majid Nili Ahmadabadi, Tehran (IR)

(21) Appl. No.: 17/129,973

(22) Filed: Dec. 22, 2020

Related U.S. Application Data
(60) Provisional application No. 62/952,314, filed on Dec. 22, 2019.

(51) Int. Cl. A61F 2/64

(52) U.S. Cl. CPC A61F 2/644; A61F 2002/5075

(57) ABSTRACT

A passive prosthetic knee may include a top adapter that may connect an exemplary passive prosthetic knee to a thigh socket and a four-link structure mimicking a knee joint attached to an exemplary top adapter. A passive prosthetic knee may further include a stance spring, a swing spring, and a switching mechanism configured to selectively urge the stance spring and the swing spring to alternately get engaged with an exemplary four-link structure. A passive prosthetic knee may further include a bottom adapter that may be utilized for connecting an exemplary passive prosthetic knee to a lower leg prosthesis. An exemplary switching mechanism may be mounted between an exemplary four-link structure and an exemplary bottom adapter.

### CROSS-REFERENCE TO RELATED APPLICATION

This application claims the benefit of priority from pending U.S. Provisional Patent Application Ser. No. 62/952,314, filed on Dec. 22, 2019, and entitled “PASSIVE PROSTHETIC KNEE WITH HYBRID PERFORMANCE,” which is incorporated herein by reference in its entirety.

### TECHNICAL FIELD

The present disclosure generally relates to prostheses and orthoses. Particularly, the present disclosure relates to prosthetic knees, and more particularly relates to a prosthetic knee for upper knee amputees.

### BACKGROUND

Prosthetic limbs are designed to help amputees restore their normal daily activities. For example, amputees who are have an upper knee amputation may require prosthetic ankle and knee to perform a normal gait cycle. A gait cycle for a leg may consist of two phases, namely a stance phase and a swing phase. In a gait cycle, the stance phase may comprise approximately 60% of the gait cycle for each leg. A stance phase for each leg may start at the instance of heel-strike and may end at the instance of toe-off of ground. Most of the energy required for performing a successful stride may be generated by Achilles muscle and ankle joint. The knee joint of each leg may carry the body weight during the stance phase and may further perform the swing phase. Hip muscles are not fully active during a swing phase and the swing phase is done by gravity acceleration. If a prosthetic knee cannot guarantee a proper swing phase, the hip muscles may be activated during the swing phase, which makes the user extremely uncomfortable.

Prosthetic legs may include active prosthetic legs and passive prosthetic legs. Active prosthetic legs may include legs that utilize motors, actuators, sensors, and micro-controllers that may control and automatically perform the stance phase and the swing phase during each gait cycle. Unfortunately, active prosthetic legs are expensive, and consequently, many users cannot afford these active prosthetic legs. Existing passive prosthetic legs may mainly include a socket, knee, shank, and foot, where the knee may be basically a hinge joint with a damper and a spring. Such simple structure of passive prosthetic knees may prevent passive prosthetic knees to emulate the normal angle of a biological knee. Consequently, conventional passive prosthetic knees cannot provide a sufficient support for users during the stance phase and furthermore they cannot provide a proper swing phase and sufficient ground clearance. Passive prosthetic legs may change a user's gait and therefore may increase their effort for having a proper swing phase. Such increased effort may lead to higher metabolic rates for the users and may further lead to heart enlargement, which is a common complication among lower limb amputees.

An intact knee, that is, a biological knee of a healthy human being without any injuries has a hybrid performance function. A hybrid performance function means that an intact knee has a high stiffness during the stance phase to support the body weight, while it has a lower stiffness during the swing phase to perform a smooth swing. An intact knee may behave like a system with instantaneous central rotation. A prosthetic knee should mimic the hybrid performance of an intact knee. Accordingly, there is a need for a high-performance passive prosthetic knee that may have a high stiffness during the stance phase and a lower stiffness during the swing phase. There is further a need for a prosthetic knee that may have a mechanism that may allow for changing the stiffness of the prosthetic knee between the stance phase and the swing phase. Additionally, it would be beneficial to have a mechanism that may allow for instantaneous central rotation.

### SUMMARY

This summary is intended to provide an overview of the subject matter of the present disclosure and is not intended to identify essential elements or key elements of the subject matter, nor is it intended to be used to determine the scope of the claimed implementations. The proper scope of the present disclosure may be ascertained from the claims set forth below in view of the detailed description and the drawings.

According to one or more exemplary embodiments, the present disclosure is directed to a passive prosthetic knee. An exemplary passive prosthetic knee may include a top adapter that may be configured to connect an exemplary passive prosthetic knee to a thigh socket. An exemplary passive prosthetic knee ma further include a four-link structure. An exemplary four-link structure may include a pair of parallel top connecting links, where each top connecting link of the pair of parallel top connecting links may be attached to a respective side of an exemplary top adapter via a top connecting plate. An exemplary four-link structure may further include a pair of parallel posterior links, where a first end of each posterior link of the pair of parallel posterior links may be pivotally connected to a first end of each respective top connecting link. An exemplary four-link structure may further include a pair of parallel bottom connecting links, where a first end of each bottom connecting link of the pair of parallel bottom connecting links may be pivotally connected to a second end of each respective posterior link. An exemplary four-link structure may further include a pair of parallel anterior links, where a first end of each anterior link of the pair of parallel anterior links may be pivotally connected to a second end of each respective top connecting link. A second end of each anterior link may further be pivotally coupled to a second end of each respective bottom connecting link.

An exemplary passive prosthetic knee may further include a stance spring that may selectively be engaged with the pair of parallel posterior links and a swing spring that may selectively be engaged with the pair of parallel posterior links. An exemplary passive prosthetic knee may further include a switching mechanism that may be configured to selectively urge an exemplary stance spring and an exemplary swing spring to alternately get engaged with an exemplary pair of parallel posterior links. Each exemplary bottom connecting link of the pair of parallel bottom connecting links may further be attached to a respective side of an exemplary switching mechanism via a middle connecting plate. An exemplary passive prosthetic knee may further include a bottom adapter that may be configured to connect an exemplary passive prosthetic knee to a lower leg prosthesis. An exemplary switching mechanism may be attached to an exemplary bottom adapter via a bottom connecting plate.

In an exemplary embodiment, an exemplary switching mechanism may include a compression spring that may be longitudinally mounted between an exemplary middle connecting plate and an exemplary bottom connecting plate. An exemplary switching mechanism may further include a spring lever, where a proximal end of an exemplary spring lever may be laterally coupled between exemplary pair of parallel posterior links via exemplary middle pivot pin holes between the first end and the second end of each posterior link of exemplary pair of parallel posterior links. An exemplary opposing distal end of an exemplary spring lever may be configured to contact an exemplary stance spring.

An exemplary switching mechanism may further include a rotatable latching member that may be laterally coupled between the second ends of pair of parallel posterior links. An exemplary rotatable latching member may be configured to selectively latch onto an exemplary spring lever to prevent the pivotal movement of an exemplary spring lever with respect to exemplary pair of parallel posterior links.

An exemplary switching mechanism may further include a double-pivot rod, where a first end of an exemplary double-pivot rod may be pivotally coupled to an exemplary bottom connecting plate. A second end of an exemplary double-pivot rod may be pivotally coupled to an exemplary rotatable latching member. An exemplary double-pivot rod may be configured to urge an exemplary rotatable latching member to latch onto an exemplary spring lever responsive to an exemplary compression spring being longitudinally compressed.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which a presently preferred embodiment of the present disclosure will now be illustrated by way of example. It is expressly understood, however, that the drawings are for illustration and description only and are not intended as a definition of the limits of the present disclosure. Embodiments of the present disclosure will now be described by way of example in association with the accompanying drawings in which:

FIG. 1 illustrates a perspective view of a passive prosthetic knee, consistent with one or more exemplary embodiments of the present disclosure;

FIG. 2 illustrates a side-view of a passive prosthetic knee, consistent with one or more exemplary embodiments of the present disclosure;

FIG. 3 illustrates an exploded view of a four-link structure of passive prosthetic knee, consistent with one or more exemplary embodiments of the present disclosure;

FIG. 4 illustrates a friction pivot axle assembly, consistent with one or more exemplary embodiments of the present disclosure;

FIG. 5 illustrates an exploded view of a stance spring, a swing spring, and a switching mechanism of a passive prosthetic knee, consistent with one or more exemplary embodiments of the present disclosure;

FIG. 6 illustrates a schematic of a gait cycle, consistent with one or more exemplary embodiments of the present disclosure;

FIGS. 7A-7C illustrate sectional side-views of a passive prosthetic knee during a stance phase of a gait cycle, consistent with one or more exemplary embodiments of the present disclosure; and

FIG. 8 illustrates a sectional side-view of a passive prosthetic knee during a swing phase of a gait cycle, consistent with one or more exemplary embodiments of the present disclosure.

### DETAILED DESCRIPTION

The novel features which are believed to be characteristic of the present disclosure, as to its structure, organization, use and method of operation, together with further objectives and advantages thereof, will be better understood from the following discussion.

The present disclosure is directed to exemplary embodiments of an exemplary passive prosthetic knee that mimics hybrid performance of an intact human knee. In other words, an exemplary passive prosthetic knee may have a high stiffness during a stance phase and a lower stiffness during a swing phase just like in an intact human knee. To this end, an exemplary passive prosthetic knee may include an exemplary stance spring with a first stiffness, an exemplary swing spring with a second stiffness, and a switching mechanism that may selectively engage one of an exemplary stance spring or an exemplary swing spring based on a phase of a gait cycle. In an exemplary embodiment, the first stiffness may be higher than the second stiffness. In an exemplary embodiment, the first stiffness may be 1.5 to 3 times stiffer than the second stiffness. An exemplary switching mechanism may be configured to identify the phase of the gait cycle, i.e., a stance phase or a swing phase. An exemplary switching mechanism may further be configured to engage either an exemplary stance spring or an exemplary swing spring based on the identified phase of the gait cycle.

An exemplary switching mechanism may be a passive mechanism that may distinguish a stance phase from a swing phase based on detecting a force exerted on an exemplary prosthetic knee due to the weight of a user. To this end, an exemplary switching mechanism may include a compression spring that upon compression under the weight of the user may urge an exemplary switching mechanism to engage an exemplary stance spring with an exemplary passive prosthetic knee. When the weight of a user is removed form an exemplary passive prosthetic knee, an exemplary compression spring may decompress and thereby urge an exemplary switching mechanism to engage an exemplary swing spring with an exemplary passive prosthetic knee.

In exemplary embodiments, such arrangement of an exemplary stance spring, an exemplary swing spring, and an exemplary switching mechanism may allow for having a stiff spring being engaged with an exemplary passive prosthetic knee during a stance phase, where the weight of a user must be stably tolerated by an exemplary passive prosthetic knee. While, during a swing phase, a relatively more flexible spring may be engaged with an exemplary passive prosthetic knee, where a smooth swinging motion of an exemplary passive prosthetic knee is required.

An exemplary passive prosthetic knee may further include a four-link structure that may allow for an easy swing initiation, great toe clearance during a swing phase and good stability during a stance phase. An exemplary four-link structure may be coupled from one end to a top adapter that may be a pyramid adapter and from an opposite end to an exemplary switching mechanism. An exemplary switching mechanism may further be coupled to a bottom adapter that may also be a pyramid adapter. An exemplary top adapter may be utilized for connecting an exemplary passive prosthetic knee to a thigh socket and an exemplary bottom adapter may be utilized for connecting an exemplary passive prosthetic knee to a lower leg prosthesis. An exemplary lower leg prosthesis may include a tube connected to a pylon. An exemplary four-link structure may mimic the functionality of a knee joint with an instantaneous center of rotation, while an exemplary switching mechanism may change a stiffness of an exemplary four-link structure by alternately engaging a stiff stance spring and a flexible swing spring as an exemplary passive prosthetic knee alternately goes through a stance phase and a swing phase of a gait cycle.

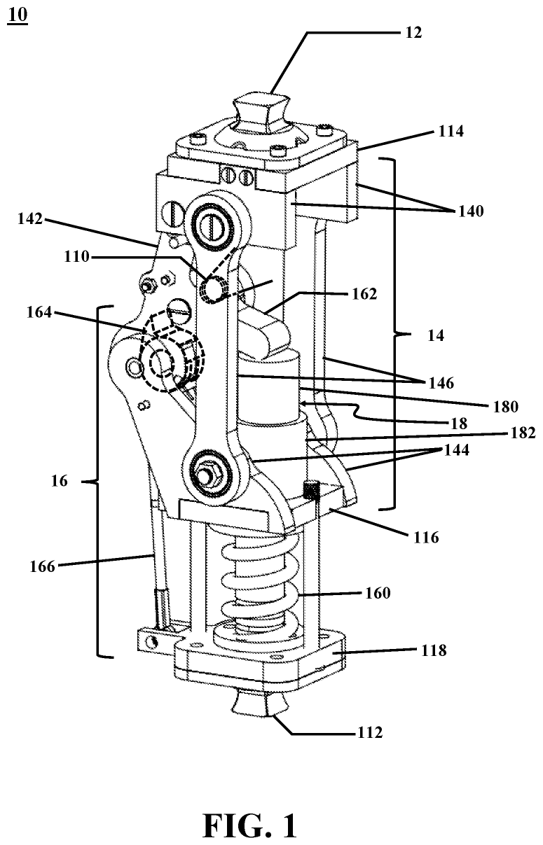

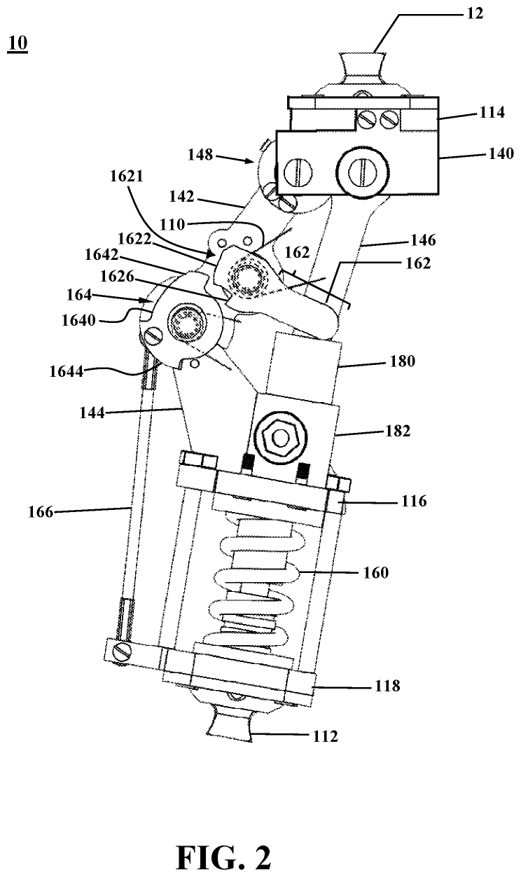

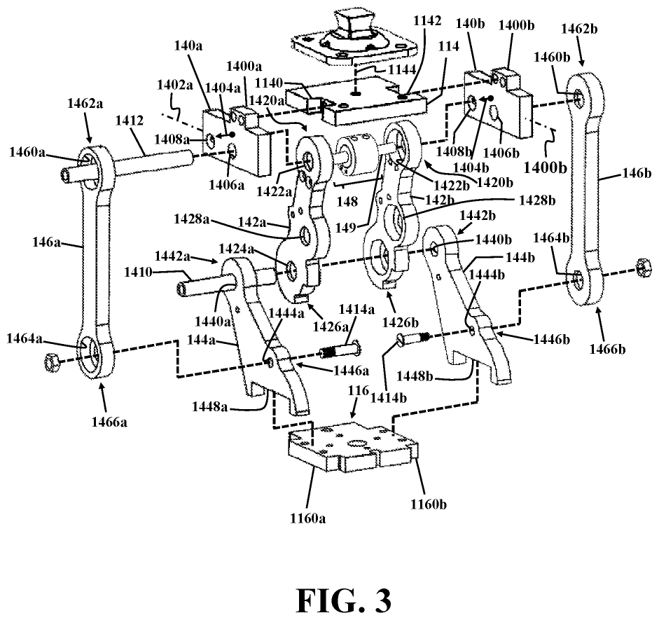

FIG. 1 illustrates a perspective view of a passive prosthetic knee 10, consistent with one or more exemplary embodiments of the present disclosure. FIG. 2 illustrates a side-view of passive prosthetic knee 10, consistent with one or more exemplary embodiments of the present disclosure. FIG. 3 illustrates an exploded view of a four-link structure of passive prosthetic knee 10, consistent with one or more exemplary embodiments of the present disclosure.

In an exemplary embodiment, passive prosthetic knee 10 may include a top adapter 12 that may be configured to connect passive prosthetic knee 10 to a thigh socket. A thigh socket is not illustrated in this figure for simplicity. In an exemplary embodiment, top adapter 12 may include a pyramid adapter or a four-prong adapter that may facilitate a connection between passive prosthetic knee 10 and a thigh socket. In an exemplary embodiment, passive prosthetic knee 10 may further include a four-link structure 14 that may be a system with an instantaneous center of rotation (ICR) similar to an intact human knee joint.

In an exemplary embodiment, four-link structure 14 may include a pair of parallel top connecting links 140, where each top connecting link of pair of parallel top connecting links 140 may be attached to a respective side of top adapter 12 via a top connecting plate 114. In an exemplary embodiment, top adapter 12 may be attached on a top surface of top connecting plate 114 and each top connecting link of pair of parallel top connecting links 140 may be attached to a respective side of top connecting plate 114. For example, parallel top connecting links 140 may include a first top connecting link 140a and a second top connecting link 140b that may include a respective first extended attachment section 1400a and a respective second extended attachment section 1400b. In an exemplary embodiment, top connecting plate 114 may further include a first recessed attachment portion 1140 and a second recessed attachment portion 1142 at respective sides of top connecting plate 114. In an exemplary embodiment, first extended attachment section 1400a may be placed within first recessed attachment portion 1140 and may be fixedly attached to first recessed attachment portion 1140 by screws or other similar fastening tools. Similarly, in an exemplary embodiment, second extended attachment section 1400b may be placed within second recessed attachment portion 1142 and may be fixedly attached to second recessed attachment portion 1142 by screws or other similar fastening tools. In an exemplary embodiment, first top connecting link 140a and second top connecting link 140b may be parallel with each other and may be symmetrically attached at respective sides of top connecting plate 114. In other words, in an exemplary embodiment, longitudinal axes of first top connecting link 140a and second top connecting link 140b may be parallel with each other. As used herein, a longitudinal axis of an object is a main axis of that object, which is associated with the longest dimension of that object. For example, a longitudinal axis 1402a of first top connecting link 140a and a longitudinal axis 1402b of second top connecting link 140b are illustrated in FIG. 3.

In an exemplary embodiment, a normal axis 1144 of top connecting plate 114 may be perpendicular to a normal axis of each top connecting link of pair of parallel top connecting links 140. As used herein, a normal axis of an object refers to an axis which is perpendicular to a plane of the largest surface of that object. For example, a normal axis 1404a of first top connecting link 140a and a normal axis 1404b of second top connecting link 140b are illustrated in FIG. 3.

In an exemplary embodiment, each top connecting link of pair of parallel top connecting links 140 may include an anterior pivot pin hole at a first end of each top connecting link of pair of parallel top connecting links 140 and a posterior pivot pin hole at a second end of each top connecting link of pair of parallel top connecting links 140. As used herein, posterior side is to the back of a user's body and anterior side is to the front of a user's body, where the front and back are defined with respect to the coronal plane of a user's body. A user may refer to a person suffering from upper knee amputation who may wear passive prosthetic knee 10.

In an exemplary embodiment, first top connecting link 140a may include a first anterior pivot pin hole 1406a and a first posterior pivot pin hole 1408a, which may be aligned with each other along longitudinal axis 1400a of first top connecting link 140a. In an exemplary embodiment, second top connecting link 140b may include a second anterior pivot pin hole 1406b and a second posterior pivot pin hole 1408b, which may be aligned with each other along longitudinal axis 1400b of second top connecting link 140b.

In an exemplary embodiment, four-link structure 14 may further include a pair of parallel posterior links 142, where a first end of each posterior link of pair of parallel posterior links 142 may be pivotally connected to the posterior pivot pin hole of each respective top connecting link of pair of top connecting links 140. For example, a first end 1420a of a first posterior link 142a of pair of parallel posterior links 142 may be pivotally connected to first posterior pivot pin hole 1408a of first top connecting link 140a and a first end 1420b of a second posterior link 142b of pair of parallel posterior links 142 may be pivotally connected to second posterior pivot pin hole 1408b of second top connecting link 140b.

In an exemplary embodiment, each posterior link of pair of parallel posterior links 142 may include a top pivot pin hole at a respective first end of each posterior link and a bottom pivot pin hole at a respective opposing second end of each posterior link. For example, first posterior link 142a may include a first top pivot pin hole 1422a at first end 1420a of first posterior link 142a and second posterior link 142b may include a second top pivot pin hole 1422b at first end 1420b of second posterior link 142b. In an exemplary embodiment, first posterior link 142a may include a first bottom pivot pin hole 1424a at a second end 1426a of first posterior link 142a and second posterior link 142b may include a second bottom pivot pin hole 1424b at a second end 1426b of second posterior link 142b.

In an exemplary embodiment, the top pivot pin hole of each posterior link of parallel posterior links 142 may be pivotally coupled with a respective posterior pivot pin hole of each respective top connecting link of pair of top connecting links 140 by a friction pivot axle assembly 148. In an exemplary embodiment, friction pivot axle assembly 148 may include a fraction pivot axle 149 coaxially mounted within a friction pivot hole 1481 of a friction pivot axle housing 1480. In an exemplary embodiment, friction pivot axle housing 1480 may be attached to the first end of each posterior link of pair of parallel posterior links 142. In an exemplary embodiment, friction pivot axle housing 1480 may be laterally attached between pair of parallel posterior links 142 with no rotational or translational movement with respect to pair of parallel posterior links 142. In an exemplary embodiment, friction pivot axle housing 1480 may be laterally attached to the first end of each posterior link of pair of parallel posterior links 142 by screws or other similar fastening tools.

In an exemplary embodiment, friction pivot hole 1481 may be laterally aligned with the top pivot pin hole of each posterior link of parallel posterior links 142 and the posterior pivot pin hole of each respective top connecting link of pair of top connecting links 140. In an exemplary embodiment, friction pivot axle 149 may be configured to pass through friction pivot hole 1481 with opposed ends of friction pivot axle 149 extending beyond friction pivot axle housing 1480 at either sides of friction pivot axle housing 1480. In an exemplary embodiment, the opposed ends of friction pivot axle 149 may be received in the top pivot pin hole of each posterior link of pair of parallel posterior links 142 and the posterior pivot pin hole of each respective top connecting link of pair of top connecting links 140. For example, first top pivot pin hole 1422a of first posterior link 142a may be aligned with first posterior pivot pin hole 1408a of first top connecting link 140a. Inner sides of first top pivot pin hole 1422a and first posterior pivot pin hole 1408a may be assembled with bearings that are not illustrated for purpose of simplicity. Such arrangement and alignment of first top pivot pin hole 1422a and first posterior pivot pin hole 1408a may allow for a first end of friction pivot axle 149 to be received in both first top pivot pin hole 1422a and first posterior pivot pin hole 1408a, thereby pivotally coupling first top pivot pin hole 1422a and first posterior pivot pin hole 1408a. Similarly, in an exemplary embodiment, a second opposing end of friction pivot axle 149 may be received in second top pivot pin hole 1422b and second posterior pivot pin hole 1408b, thereby pivotally coupling second top pivot pin hole 1422b and second posterior pivot pin hole 1408b. In an exemplary embodiment, pair of parallel posterior links 142 may pivot about a longitudinal axis of friction pivot axle 149 with respect to pair of top connecting links 140. As used herein, the longitudinal axis of friction pivot axle 149 may refer to a main axis of friction pivot axle 149 associated with the longest dimension of friction pivot axle 149.

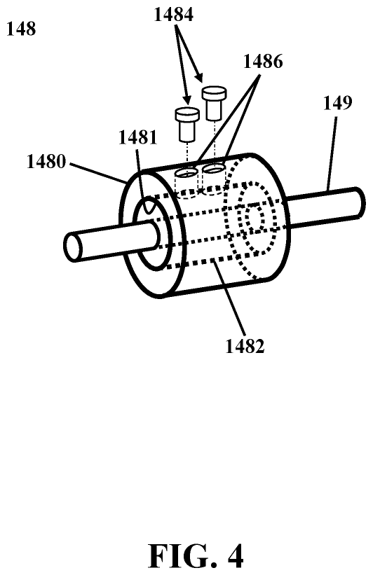

FIG. 4 illustrates friction pivot axle assembly 148, consistent with one or more exemplary embodiments of the present disclosure. In an exemplary embodiment, friction pivot axle assembly 148 may further include a resilient friction pad 1482 that may be coaxially disposed within friction pivot hole 1481 of friction pivot axle housing 1480. In an exemplary embodiment, resilient friction pad 1482 may surround friction pivot axle 149 and may engage an outer surface of friction pivot axle 149 with an adjustable resilience.

In an exemplary embodiment, friction pivot axle assembly 148 may further include one or more adjustment screws 1484 that may be mounted on friction pivot axle housing 1480 in contact with resilient friction pad 1482. In an exemplary embodiment, adjustment screws 1484 may be configured to adjust the adjustable resilience of resilient friction pad 1482. As used herein, adjusting the adjustable resilience may refer to adjusting the damping force exerted by resilient friction pad 1482 onto friction pivot axle 149. In an exemplary embodiment, a damping force acting on friction pivot axle 149 may be increased by pressing resilient friction pad 1482 on the outer surface of friction pivot axle 149 responsive to adjustment screws 1484 being turned in a first direction. While a damping force acting on friction pivot axle 149 may be decreased by releasing resilient friction pad 1482 responsive to adjustment screws 1484 being turned in a second opposite direction.

In an exemplary embodiment, friction pivot axle housing 1480 may include one or more corresponding screw holes 1486 that may be configured for receiving adjustment screws 1484. In an exemplary embodiment, screw holes 1486 may include holes on an outer surface of friction pivot axle housing 1480 that may extend downward into friction pivot hole 1481 providing access to the outer surface of resilient friction pad 1482. In an exemplary embodiment, adjustment screws 1484 may be screwed in or out of screw holes 1486. For example, adjustment screws 1484 may be screwed in screw holes 1486, responsive to adjustment screws 1484 being turned in the first direction and adjustment screws 1484 may be screwed out of screw holes 1486, responsive to adjustment screws 1484 being turned in the second direction.

In an exemplary embodiment, four-link structure 14 may further include a pair of parallel bottom connecting links 144, where a first end of each bottom connecting link of pair of parallel bottom connecting links 144 may be pivotally connected to a second end of each respective posterior link of pair of parallel posterior links 142. In an exemplary embodiment, each bottom connecting link of pair of parallel bottom connecting links 144 may include a top pivot pin hole at the first end of each bottom connecting link and a bottom pivot pin hole at the opposing second end of each bottom connecting link. For example, a first bottom connecting link 144a of pair of parallel bottom connecting links 144 may include a first top pivot pin hole 1440a at a first end 1442a of first bottom connecting link 144a. A first bottom connecting link 144a of pair of parallel bottom connecting links 144 may further include a first bottom pivot pin hole 1444a at a second end 1446a of first bottom connecting link 144a. Similarly, a second bottom connecting link 144b of pair of parallel bottom connecting links 144 may include a second top pivot pin hole 1440b at a first end 1442b of second bottom connecting link 144b. A second bottom connecting link 144b of pair of parallel bottom connecting links 144 may further include a second bottom pivot pin hole 1444b at a second end 1446a of second bottom connecting link 144b.

In an exemplary embodiment, the top pivot pin hole of each bottom connecting link of pair of parallel bottom connecting links 144 may be pivotally connected to the bottom pivot pin hole of each respective posterior link of pair of parallel posterior links 142 by a first pivot axle 1410. For example, first top pivot pin hole 1440a of first bottom connecting link 144a may be pivotally coupled with first bottom pivot pin hole 1424a of first posterior link 142a. In an exemplary embodiment, first top pivot pin hole 1440a and first bottom pivot pin hole 1424a may be assembled with bearings that may allow for first pivot axle 1410 to be rotatably received within first top pivot pin hole 1440a and first bottom pivot pin hole 1424a, thereby pivotally coupling first top pivot pin hole 1440a and first bottom pivot pin hole 1424a such that first posterior link 142a may pivot about a longitudinal axis of first pivot axle 1410 with respect to first bottom connecting link 144a.

Similarly, second top pivot pin hole 1440b of second bottom connecting link 144b may be pivotally coupled with second bottom pivot pin hole 1424b of second posterior link 142b. In an exemplary embodiment, second top pivot pin hole 1440b and second bottom pivot pin hole 1424b may be assembled with bearings that may allow for first pivot axle 1410 to be rotatably received within second top pivot pin hole 1440b and second bottom pivot pin hole 1424b, thereby pivotally coupling second top pivot pin hole 1440b and second bottom pivot pin hole 1424b such that second posterior link 142b may pivot about the longitudinal axis of first pivot axle 1410 with respect to second bottom connecting link 144b.

In an exemplary embodiment, each bottom connecting link of pair of parallel bottom connecting links 144 may further include a respective recessed bottom connection portion at the second end of each bottom connecting link of pair of parallel bottom connecting links 144. In an exemplary embodiment, each respective recessed bottom connection portion of each bottom connecting link may be attached to a respective side of a middle connecting plate 116. In an exemplary embodiment, middle connecting plate 116 may be a plate parallel with top connecting plate 114. In an exemplary embodiment, middle connecting plate 116 may include protruding connecting portions at respective sides of middle connecting plate 116, where respective recessed bottom connection portion of each bottom connecting link may be attached to a respective protruding connecting portions of middle connecting plate 116. For example, first bottom connecting link 144a may include a first recessed bottom connecting portion 1448a that may be attached to a first protruding connecting portion 1160a of middle connecting plate 116. Similarly, second bottom connecting link 144b may include a second recessed bottom connecting portion 1448b that may be attached to a second protruding connecting portion 1160b of middle connecting plate 116. In an exemplary embodiment, first bottom connecting link 144a and second bottom connecting link 144b may be fixedly attached to respective sides of middle connecting plate 116, such that first bottom connecting link 144a and second bottom connecting link 144b may have no rotational or translational motion with respect to middle connecting plate 116.

In an exemplary embodiment, four-link structure 14 may further include a pair of parallel anterior links 146. In an exemplary embodiment, a first end of each anterior link of pair of parallel anterior links 146 may be pivotally connected to a second end of each respective top connecting link of pair of top connecting links 140. In an exemplary embodiment, a second end of each anterior link pair of parallel anterior links 146 may be pivotally coupled to a second end of each respective bottom connecting link of pair of parallel bottom connecting links 144.

In an exemplary embodiment, each anterior link of pair of parallel anterior links 146 may include a top pivot pin hole at the first end of each anterior link of pair of parallel anterior links 146 and a bottom pivot pin hole at the opposing second end of each anterior link of pair of parallel anterior links 146. In an exemplary embodiment, the top pivot pin hole of each anterior link of pair of parallel anterior links 146 may be pivotally connected to the anterior pivot pin hole of each respective top connecting link of pair of top connecting links 140 by a second pivot axle 1412. For example, a first anterior link 146a of pair of parallel anterior links 146 may include a first top pivot pin hole 1460a at a first end 1462a of first anterior link 146a. In an exemplary embodiment, first top pivot pin hole 1460a may be aligned with first anterior pivot pin hole 1406a such that second pivot axle 1412 may pass through both first top pivot pin hole 1460a and first anterior pivot pin hole 1406a. In an exemplary embodiment, first top pivot pin hole 1460a may be assembled with a bearing that may allow for a pivotal movement of first anterior link 146a about a longitudinal axis of second pivot axle 1412 with respect to first top connecting link 140a. Similarly, a second anterior link 146b of pair of parallel anterior links 146 may include a second top pivot pin hole 1460b at a first end 1462b of second anterior link 146b. In an exemplary embodiment, second top pivot pin hole 1460b may be aligned with second anterior pivot pin hole 1406b such that second pivot axle 1412 may pass through both second top pivot pin hole 1460b and second anterior pivot pin hole 1406b. In an exemplary embodiment, second top pivot pin hole 1460b may be assembled with a bearing that may allow for a pivotal movement of second anterior link 146b about a longitudinal axis of second pivot axle 1412 with respect to second top connecting link 140b.

In an exemplary embodiment, the bottom pivot pin hole of each anterior link of pair of parallel anterior links 146 may be pivotally coupled to the bottom pivot pin hole of each respective bottom connecting link of pair of parallel bottom connecting links 144 by a respective pivot pin of a pair of pivot pins. For example, first anterior link 146a may include a first bottom pivot pin hole 1464a at a second end 1466a of first anterior link 146a. In an exemplary embodiment, first bottom pivot pin hole 1464a may be aligned with first bottom pivot pin hole 1444a of first bottom connecting link 144a such that a first pivot pin 1414a may pass through both first bottom pivot pin hole 1464a and first bottom pivot pin hole 1444a. In an exemplary embodiment, first bottom pivot pin hole 1464a may be assembled with a bearing that may allow for a pivotal movement of first anterior link 146a about a longitudinal axis of first pivot pin 1414a with respect to first bottom connecting link 144a. Similarly, second anterior link 146b may include a second bottom pivot pin hole 1464b at a second end 1466b of second anterior link 146b. In an exemplary embodiment, second bottom pivot pin hole 1464b may be aligned with second bottom pivot pin hole 1444b of second bottom connecting link 144b such that a second pivot pin 1414b may pass through both second bottom pivot pin hole 1464b and second bottom pivot pin hole 1444b. In an exemplary embodiment, second bottom pivot pin hole 1464b may be assembled with a bearing that may allow for a pivotal movement of second anterior link 146b about a longitudinal axis of second pivot pin 1414b with respect to second bottom connecting link 144b.

FIG. 2 illustrates the side-view of passive prosthetic knee 10 without one of posterior links of pair of parallel posterior links 142, one of bottom connecting links of pair of parallel bottom connecting links 144, and one of anterior links of pair of parallel anterior links 146 for purpose of simplicity. Such removal of one of posterior links of pair of parallel posterior links 142, one of bottom connecting links of pair of parallel bottom connecting links 144, and one of anterior links of pair of parallel anterior links 146 from this figure allows for other inner elements of passive prosthetic knee 10 to be visible.

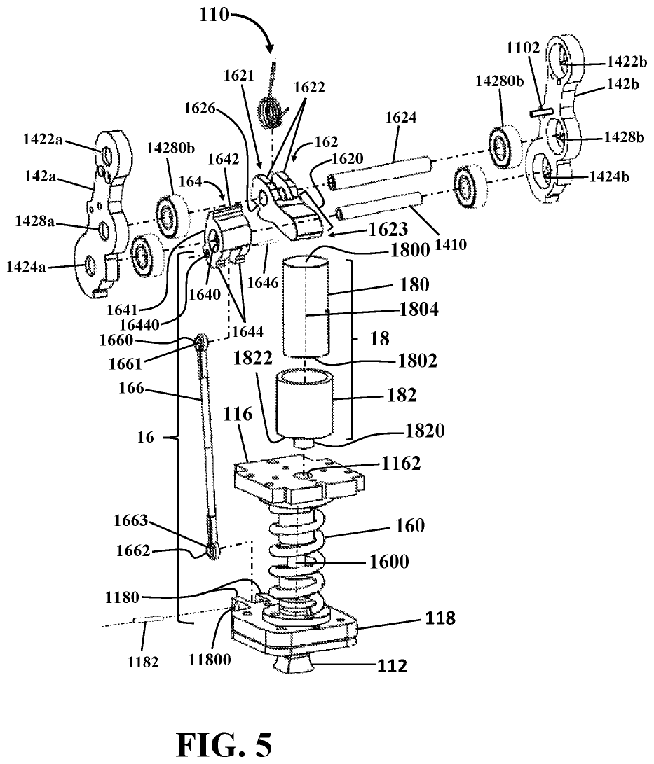

FIG. 5 illustrates an exploded view of a stance spring 18, a swing spring 110, and a switching mechanism 16 of passive prosthetic knee 10, consistent with one or more exemplary embodiments of the present disclosure. In an exemplary embodiment, stance spring 18 may be mounted on middle connecting plate 116. In an exemplary embodiment, stance spring 18 may include a resilient elongated body 180 that may longitudinally extend between a top end 1800 of the resilient elongated body 180 and a bottom end 1802 of resilient elongated body 180. In an exemplary embodiment, a longitudinal axis 1804 of resilient elongated body 180 may be parallel with a normal axis of middle connecting plate 116. In an exemplary embodiment, resilient elongated body 180 may be made of a polyurethane material in a cylindrical configuration. In an exemplary embodiment, resilient elongated body 180 may have the characteristics of a coil spring in that resilient elongated body 180 may be capable of resiliently compressing along longitudinal axis 1804 and of springing back to an original form of resilient elongated body 180.

In an exemplary embodiment, stance spring 18 may further include a rigid elongated housing 182 that may be attached on middle connecting plate 116. In an exemplary embodiment, middle connecting plate 116 may include a threaded hole 1162 and rigid elongated housing 182 may include a bottom extended threaded portion 1820 that may be screwed into threaded hole 1162 of middle connecting plate 116. In an exemplary embodiment, resilient elongated body 180 may be disposed within rigid elongated housing 182 such that bottom end 1802 of resilient elongated body 180 may abut against a base end 1822 of rigid elongated housing 182 while top end 1800 of resilient elongated body 180 may extend out of rigid elongated housing 182. In an exemplary embodiment, rigid elongated housing 182 may be an annular body that may extend upward along the normal axis of middle connecting plate 116 and resilient elongated body 180 may coaxially be disposed within rigid elongated housing 182.

In an exemplary embodiment, passive prosthetic knee 10 may further include switching mechanism 16 that may be configured to selectively urge stance spring 18 and swing spring 110 to alternately get engaged with pair of parallel posterior links 142. In an exemplary embodiment, switching mechanism 16 may include a compression spring 160 that may be longitudinally mounted between middle connecting plate 116 and a bottom connecting plate 118. In an exemplary embodiment, bottom connecting plate 118 may be parallel with middle connecting plate 116 and top connecting plate 114. In an exemplary embodiment, a bottom adapter 112 may be attached beneath bottom connecting plate 118, where bottom adapter 112 may be configured to connect the passive prosthetic knee 10 to a lower leg prosthesis. A lower leg prosthesis is not illustrated for simplicity. In an exemplary embodiment, bottom adapter 112 may include a pyramid adapter or a four-prong adapter that may facilitate a connection between passive prosthetic knee 10 and a lower leg prosthesis.

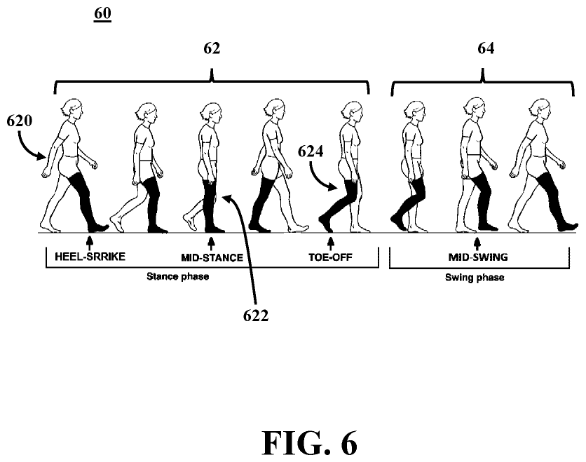

FIG. 6 illustrates a schematic of a gait cycle 60, consistent with one or more exemplary embodiments of the present disclosure. Gait cycle 60 has two phases, namely, a stance phase 62 and a swing phase 64. Stance phase 62 may begin at the moment of heel-strike (620) and then continues through mid-stance (622) and ends at the moment of toe-off (624). After the toe-off moment (624), swing phase 64 begins, where the leg is swung toward the next gait cycle.

In an exemplary embodiment, compression spring 160 may include a spring such as an open-coil helical spring wound around an axis of wind 1600. In an exemplary embodiment, compression spring 160 may oppose compression along axis of wind 1600. At the moment of heel-strike (620) in stance phase 62 when the heel strikes the ground, compression spring 160 may be compressed and shortened under the weight of a user. Compression spring 160 may remain compressed during mid-stance (622) until the moment of toe-off (624), which is the end of stance phase 62 and beginning of swing phase 64 of gait cycle 60. In other words, compression spring 160 may be configured to be compressed during stance phase 62 of gait cycle 60.

In an exemplary embodiment, switching mechanism 16 may further include a spring lever 162, where a proximal end 1621 of spring lever 162 may be laterally coupled between pair of parallel posterior links 142. In an exemplary embodiment, each posterior link of pair of parallel posterior links 142 may include a respective middle pivot pin hole that may be positioned between respective top pivot pin hole and bottom pivot pin hole of each posterior link of pair of parallel posterior links 142. For example, first posterior link 142a may include a first middle pivot pin hole 1428a that may be positioned on first posterior link 142a above first bottom pivot pin hole 1424a and below first top pivot pin hole 1422a. Similarly, second posterior link 142b may include a second middle pivot pin hole 1428b that may be positioned on second posterior link 142b above second bottom pivot pin hole 1424b and below second top pivot pin hole 1422b.

In an exemplary embodiment, spring lever 162 may include an extended part 1620 that may extend between proximal end 1621 and distal end 1623 of spring lever 162. Extended part 1620 may abut against top end 1800 of resilient elongated body 180 at distal end 1623 of spring lever 162. In an exemplary embodiment, spring lever 162 may further include a pair of parallel flanges 1622 at proximal end 1621 of spring lever 162, where each flange of pair of parallel flanges 1622 may include a respective pivot pin hole. A respective pivot pin hole of each flange of pair of parallel flanges 1622 may be configured to receive opposed ends of a pivot pin 1624, which may pass through the respective middle pivot pin holes of pair of parallel posterior links 142. For example, first middle pivot pin hole 1428a may be assembled with a first bearing 14280a and second middle pivot pin hole 1428b may be assembled with a second bearing 14280b that may allow for pivot pin 1624 to be pivotally coupled laterally between first posterior link 142a and second posterior link 142b. In an exemplary embodiment, spring lever 162 may pivot relative to pair of parallel posterior links 142 at proximal end 1621 of spring lever 162 about a longitudinal axis of pivot pin 1624.

In an exemplary embodiment, switching mechanism 16 may further include a rotatable latching member 164 that may be laterally coupled between the second ends of pair of parallel posterior links 142. For example, rotatable latching member 164 may include a pivot pin hole 1640 that may be configured to allow first pivot axle 1410 to pass through and be rotatably coupled with rotatable latching member 164. In an exemplary embodiment, such coupling between rotatable latching member 164 and first pivot axle 1410 may allow for rotatably coupling rotatable latching member 164 with first lower pivot pin hole 1424a of first posterior link 142a from one side and with second lower pivot pin hole 1424b of second posterior link 142b from the other opposing side of rotatable latching member 164. In an exemplary embodiment, rotatable latching member 164 may be configured to selectively latch onto spring lever 162 to prevent the pivotal movement of spring lever 162 with respect to pair of parallel posterior links 142. As used herein, selectively latching may refer to rotatable latching member 164 being configured to latch onto spring lever 162 responsive to compression spring 160 being compressed and shortened under the weight of a user during stance phase 62.

In an exemplary embodiment, switching mechanism 16 may further include a double-pivot rod 166. In an exemplary embodiment, a first end 1660 of double-pivot rod 166 may be pivotally coupled to bottom connecting plate 118 and a second end 1662 of double-pivot rod 166 may be pivotally coupled to rotatable latching member 164. In an exemplary embodiment, double-pivot rod 166 may be configured to urge rotatable latching member 164 to latch onto spring lever 162 responsive to compression spring 160 being longitudinally compressed under the weight of a user during stance phase 62.

In an exemplary embodiment, rotatable latching member 164 may further include a main body 1641, through which pivot pin hole 1640 may be provided, a latching tongue 1642 protruded from main body 1640, and a first pair of parallel flanges 1644 that may extend from main body 1641. In an exemplary embodiment, latching tongue 1642 may be configured to engage spring lever 162 responsive to main body 1641 pivoting about first pivot axle 1410 in a first direction. In an exemplary embodiment, latching tongue 1642 may further be configured to release spring lever 162 responsive to main body 1641 pivoting about first pivot axle 1410 in a second opposing direction. In an exemplary embodiment, spring lever 162 may further include a recessed portion 1626 at proximal end 1621 of spring lever 162. In an exemplary embodiment, recessed portion 1626 may be configured to receive latching tongue 1642 within recessed portion 1626. As used herein, latching tongue 1642 engaging spring lever 162 may refer to latching tongue 1642 being stuck within recessed portion 1626 of spring lever 162.

In an exemplary embodiment, each flange of first pair of parallel flanges 1644 may include a respective first pivot pin hole, such as pivot pin hole 16440. The respective first pivot pin hole of each flange of first pair of parallel flanges 1644 may be configured to receive opposed ends of a first pivot pin 1646 passing through a proximal pivot pin hole 1661 at first end 1660 of double-pivot rod 166.

In an exemplary embodiment, bottom connecting plate 118 may further include a second pair of parallel flanges 1180. In an exemplary embodiment, each flange of second pair of parallel flanges 1180 may include a second pivot pin hole, such as pivot pin hole 11800. The second pivot pin hole of each flange of parallel flanges 1180 may be configured to receive opposed ends of a second pivot pin 1182 passing through a distal pivot pin hole 1663 at second end 1662 of double-pivot rod 166.

In an exemplary embodiment, such pivotal connection of double-pivot rod 166 to bottom connecting plate 118 form second end 1662 and to rotatable latching member 164 from first end 1660 of double-pivot rod 166 may allow for double-pivot rod 166 to urge main body 1641 of rotatable latching member 164 to pivot about first pivot axle 1410 in a first direction in response to compression spring 160 being longitudinally compressed between middle connecting plate 116 and bottom connecting plate 116. In an exemplary embodiment, latching tongue 1642 may move into and engage with recessed portion 1626 at proximal end 1621 of spring lever 162 responsive to main body 1641 pivoting about first pivot axle 1410 in the first direction.

In an exemplary embodiment, such pivotal connection of double-pivot rod 166 to bottom connecting plate 118 form second end 1662 and to rotatable latching member 164 from first end 1660 of double-pivot rod 166 may further allow for double-pivot rod 166 to urge main body 1641 of rotatable latching member 164 to pivot about first pivot axle 1410 in a second direction in response to compression spring 160 being longitudinally decompressed at the end of stance phase 62 and beginning of swing phase 64. In an exemplary embodiment, latching tongue 1642 may move out of and disengage recessed portion 1626 at proximal end 1621 of spring lever 162 responsive to main body 1641 pivoting about first pivot axle 1410 in the second direction.

In an exemplary embodiment, swing spring 110 may include a torsion coil spring that may be mounted around pivot pin 1624. In an exemplary embodiment, swing spring 110 may be laterally disposed between pair of parallel flanges 1622 of spring lever 162 and may be rotatable with spring lever 162 about the longitudinal axis of pivot pin 1624. In an exemplary embodiment, a first rod 1102 may be attached between pair of parallel posterior links 142 above first and second middle pivot pin hole (1428a, 1428b), upon which swing spring 110 may be engaged during swing phase 64.

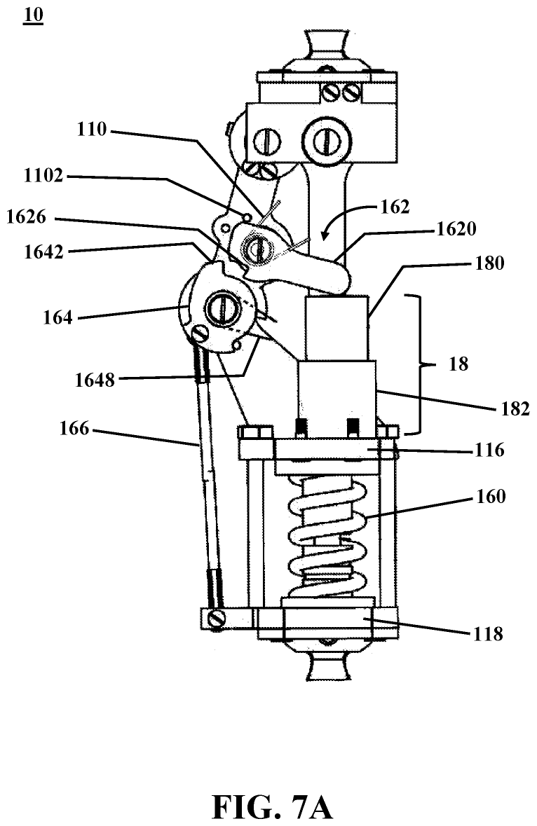

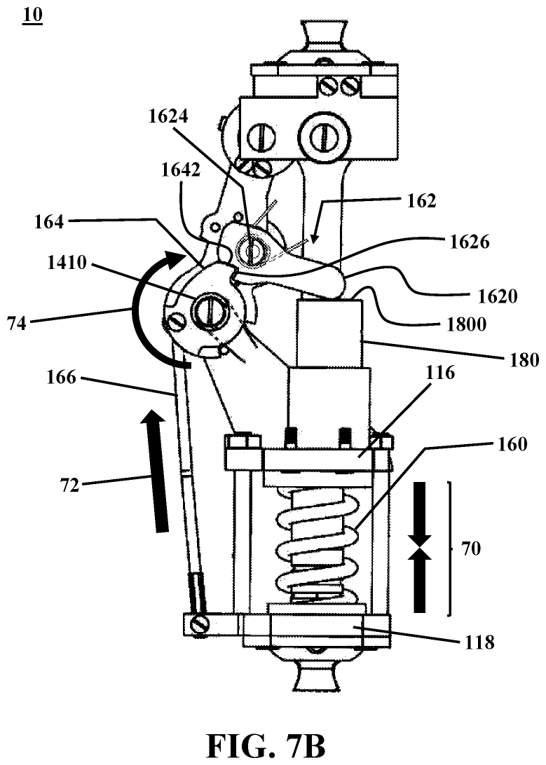

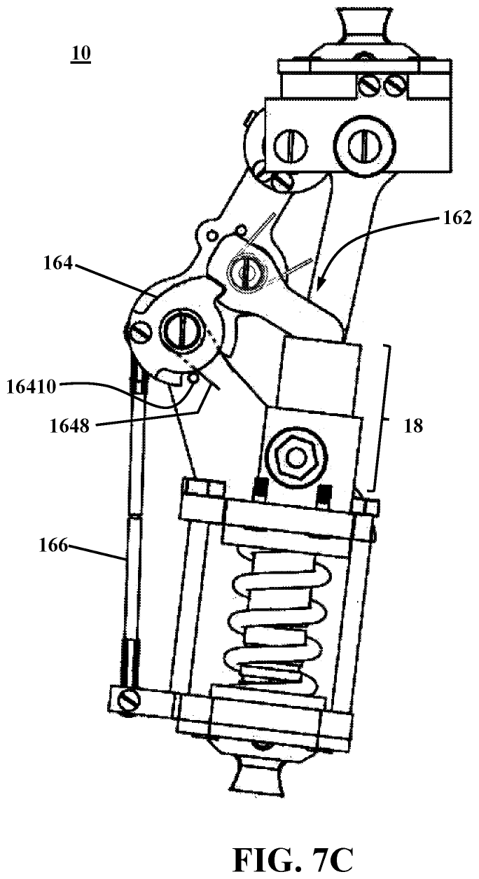

FIGS. 7A-7C illustrate sectional side-views of passive prosthetic knee 10 during a stance phase of a gait cycle, consistent with one or more exemplary embodiments of the present disclosure. For purpose of simplicity, one of posterior links of pair of parallel posterior links 142, one of pair of parallel anterior links 146, and one of pair of parallel bottom connecting links 144 are removed from these figures to avoid obstruction of other internal parts of passive prosthetic knee 10.

FIG. 7A illustrates a sectional side-view of passive prosthetic knee 10 before heel strike at the beginning of a stance phase of a gait cycle, consistent with one or more exemplary embodiments of the present disclosure. In an exemplary embodiment, before the moment of heel strike, compression spring 160 may be in its decompressed state between middle connecting plate 116 and bottom connecting plate 118.

FIG. 7B illustrates a sectional side-view of passive prosthetic knee 10 after heel strike at the beginning of a stance phase of a gait cycle, consistent with one or more exemplary embodiments of the present disclosure. In an exemplary embodiment, after heel strike and the beginning of the stance phase of the gait cycle, compression spring 160 may be compressed between middle connecting plate 116 and bottom connecting plate 118 under the weight of a user. In an exemplary embodiment, compression of compression spring 160 and shortening of its length along a direction shown by arrows 70 may urge double-pivot rod 166 to move upwardly along a direction shown by arrow 72. In an exemplary embodiment, pivotal coupling of double-pivot rod 166 with rotatable latching member 164 may allow for conversion of the linear movement of double-pivot rod 166 along the direction shown by arrow 72 to a rotational movement of rotatable latching member 164 about longitudinal axis of first pivot axle 1410 in a first direction 74. As discussed before, the rotational movement of rotatable latching member 164 about longitudinal axis of first pivot axle 1410 in first direction 74 may place latching tongue 1642 within recessed portion 1626 of spring lever 162 and thereby prevents any rotational movements of spring lever 162 about the longitudinal axis of pivot pin 1624. In an exemplary embodiment, extended part 1620 may abut against top end 1800 of resilient elongated body 180 and during the stance phase of the gait cycle, when spring lever 162 may be locked by rotatable latching member 164.

FIG. 7C illustrates a sectional side-view of passive prosthetic knee 10 during mid-stance to toe off moment of a stance phase of a gait cycle, consistent with one or more exemplary embodiments of the present disclosure. In an exemplary embodiment, spring lever 162 may remain engaged with stance spring 18 during the entire stance phase from the moment of heel strike to the moment of toe off. In an exemplary embodiment, such engagement of spring lever 162 and stance spring 18 may allow for supporting the weight of the user on stance spring 18.

In an exemplary embodiment, rotatable latching member 164 may further include a restoring spring 1648 that may be a torsion coil spring. In an exemplary embodiment, a second rod 16410 may be attached between pair of bottom connecting links 144 below first and second top pivot pin holes (1440a and 1440b), upon which restoring spring 1648 may be engaged. In an exemplary embodiment, such engagement of restoring spring 1648 and second rod 16410 may allow for rotatable latching member 164 to restore its initial position when double-pivot rod 166 is not urging rotatable latching member 164 to rotate in first direction 74, a situation which is discussed further in the following paragraph.

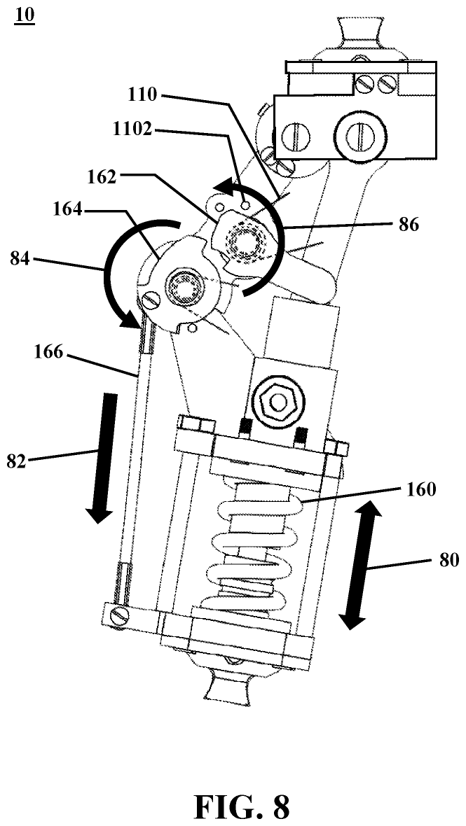

FIG. 8 illustrates a sectional side-view of passive prosthetic knee 10 during a swing phase of a gait cycle, consistent with one or more exemplary embodiments of the present disclosure. In an exemplary embodiment, after the toe-off moment when the stance phase of a gait cycle ends and the swing phase starts, the weight of the user is removed from passive prosthetic knee 10 and now the leg is about to swing into the next stance phase. During the swing phase, the weight of the user is removed from compression spring 160 and as a result, compression spring may be decompressed, and its length may be extended along a direction shown by arrow 80. In an exemplary embodiment, as compression spring 160 decompresses, double-pivot rod 166 may be urged to move downwardly in a direction shown by arrow 82. The downward movement of double-pivot rod 166 may in turn urge rotatable latching member 164 to pivot about the longitudinal axis of first pivot axle 1410 in a second direction 84. In an exemplary embodiment, such pivotal movement of rotatable latching member 164 in second direction 84, which is further encouraged by restoring spring 1648, as discussed in the preceding paragraph, may release spring lever 162. In an exemplary embodiment, when spring lever 162 is not engaged with rotatable latching member 164, extended part 1620 of spring lever 162 may not be firmly abutting against top end 1800 of stance spring 18, instead it may pivot along a direction shown by arrow 86. In an exemplary embodiment, swing spring 110 may pivot along the direction shown by arrow 86 with stance spring 18 to a point where swing spring 110 may engage with first rod 1102. In an exemplary embodiment, engagement of swing spring 110 and first rod 1102 may allow for swing spring 110 to be engaged with four-link structure 14 during the swing phase.

In an exemplary embodiment, such arrangement of stance spring 18, swing spring 110, and the switching mechanism may allow for four-link structure 14 to bend under the weight of a user during stance phase 62 with a first stiffness equal to the stiffness of stance spring 18. In an exemplary embodiment, such arrangement of stance spring 18, swing spring 110, and the switching mechanism may further allow for four-link structure 14 to bend during swing phase 64 with a second stiffness equal to the stiffness of swing spring 110. In an exemplary embodiment, the first stiffness may be larger in comparison with the second stiffness. In an exemplary embodiment, such mechanical structure of the switching mechanism with no active components, such as motors and electrical subsystems, may reduce the implementation and maintenance costs of an exemplary passive prosthetic knee, while enabling an exemplary passive prosthetic knee to have similar capabilities of an active prosthetic knee.

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for description and not of limitation, such that the terminology or phraseology of the present specification is to be interpreted by the skilled artisan in light of the teachings and guidance.

The breadth and scope of the present disclosure should not be limited by any of the above-described exemplary embodiments but should be defined only in accordance with the following claims and their equivalents.

Throughout this specification and the claims which follow, unless the context requires otherwise, the word “comprise”, and variations such as “comprises” or “comprising”, will be understood to imply the inclusion of a stated integer or step or group of integers or steps but not to the exclusion of any other integer or step or group of integers or steps.

Moreover, the word “substantially” when used with an adjective or adverb is intended to enhance the scope of the particular characteristic, e.g., substantially planar is intended to mean planar, nearly planar and/or exhibiting characteristics associated with a planar element. Further use of relative terms such as “vertical”, “horizontal”, “up”, “down”, and “side-to-side” are used in a relative sense to the normal orientation of the apparatus.

What is claimed is:

1. A passive prosthetic knee, comprising:
a top adapter configured to connect the passive prosthetic knee to a thigh socket;
a four-link structure comprising:
a pair of parallel top connecting links, each top connecting link of the pair of parallel top connecting links attached to a respective side of the top adapter via a top connecting plate;
a pair of parallel posterior links, a first end of each posterior link of the pair of parallel posterior links pivotally connected to a first end of each respective top connecting link;
a pair of parallel bottom connecting links, a first end of each bottom connecting link of the pair of parallel bottom connecting links pivotally connected to a second end of each respective posterior link; and
a pair of parallel anterior links, a first end of each anterior link of the pair of parallel anterior links pivotally connected to a second end of each respective top connecting link, a second end of each anterior link pivotally coupled to a second end of each respective bottom connecting link;
a stance spring selectively engaged with the pair of parallel posterior links;
a swing spring selectively engaged with the pair of parallel posterior links;
a switching mechanism configured to selectively urge the stance spring and the swing spring to alternately get engaged with the pair of parallel posterior links, each bottom connecting link of the pair of parallel bottom connecting links further attached to a respective side of the switching mechanism via a middle connecting plate; and
a bottom adapter configured to connect the passive prosthetic knee to a lower leg prosthesis, the switching mechanism attached to the bottom adapter via a bottom connecting plate,
wherein the switching mechanism comprises:
a compression spring longitudinally mounted between the middle connecting plate and the bottom connecting plate;
a spring lever, a proximal end of the spring lever laterally coupled between the pair of parallel posterior links via middle pivot pin holes between the first end and the second end of each posterior link of the pair of parallel posterior links, an opposing distal end of the spring lever configured to contact the stance spring;
a rotatable latching member laterally coupled between the second ends of pair of parallel posterior links, the rotatable latching member configured to selectively latch onto the spring lever to prevent the pivotal movement of the spring lever with respect to the pair of parallel posterior links; and
a double-pivot rod, a first end of the double-pivot rod pivotally coupled to the bottom connecting plate, a second end of the double-pivot rod pivotally coupled to the rotatable latching member, the double-pivot rod configured to urge the rotatable latching member to latch onto the spring lever responsive to the compression spring being longitudinally compressed.

2. The passive prosthetic knee of claim 1, wherein the stance spring comprises:
a resilient elongated body longitudinally extended between a top end of the resilient elongated body and a bottom end of the resilient elongated body, the top end of the resilient elongated body in contact with the spring lever; and
a rigid elongated housing attached on the middle connecting plate, the resilient elongated body disposed within the rigid elongated housing, the bottom end of the resilient elongated body abutting against a base end of the rigid elongated housing, the top end of the resilient elongated body extended out of the rigid elongated housing.

3. The passive prosthetic knee of claim 2, wherein the spring lever comprises:
an extended part extended between the proximal end and the distal end of the spring lever, the extended part abutting against the top end of the resilient elongated body; and
a pair of parallel flanges at the proximal end of the spring lever, each flange of the pair of parallel flanges comprising a pivot pin hole, the pivot pin hole of each flange configured to receive opposed end of a pivot pin passing through the middle pivot pin holes of the pair of parallel posterior links, the spring lever pivotable relative to the pair of parallel posterior links at the proximal end of the spring lever about the pivot pin.

4. The passive prosthetic knee of claim 3, wherein the swing spring comprises a torsion coil spring mounted around the pivot pin, the torsion coil spring laterally disposed between the pair of parallel flanges.

5. The passive prosthetic knee of claim 4, wherein each top connecting link of the pair of parallel top connecting links comprises an anterior pivot pin hole at the first end of each top connecting link of the pair of parallel top connecting links and a posterior pivot pin hole at the second end of each top connecting link of the pair of parallel top connecting links.

6. The passive prosthetic knee of claim 5, wherein each posterior link of the pair of parallel posterior links comprising a top pivot pin hole at the first end of each posterior link and a bottom pivot pin hole at the opposing second end of each posterior link, the top pivot pin hole of each posterior link pivotally coupled with the posterior pivot pin hole of each respective top connecting link by a friction pivot axle.

7. The passive prosthetic knee of claim 6, wherein the friction pivot axle comprises:
a friction pivot axle housing comprising a friction pivot hole, the friction pivot axle housing attached to the first end of each posterior link, the friction pivot axle housing laterally attached between the pair of parallel posterior links, the friction pivot hole aligned with the top pivot pin hole of each posterior link and the posterior pivot pin hole of each respective top connecting link, the friction pivot axle configured to pass through the a friction pivot hole with opposed ends of the friction pivot axle extending beyond the friction pivot axle housing at either sides of the friction pivot axle housing, the opposed ends of the friction pivot axle received in the top pivot pin hole of each posterior link and the posterior pivot pin hole of each respective top connecting link;
a resilient friction pad coaxially disposed within the friction pivot hole of the friction pivot axle housing, the resilient friction pad surrounding the friction pivot axle, the resilient friction pad engaging an outer surface of the friction pivot axle with an adjustable resilience; and
an adjustment screw mounted on the friction pivot axle housing in contact with the resilient friction pad, the adjustment screw configured to adjust the adjustable resilience of the resilient friction pad by pressing the resilient friction pad on the outer surface of the friction pivot axle responsive to the adjustment screw being turned in a first direction, the adjustment screw further configured to adjust the adjustable resilience of the resilient friction pad by releasing the resilient friction pad responsive to the adjustment screw being turned in a second opposite direction.

8. The passive prosthetic knee of claim 6, wherein each bottom connecting link of the pair of parallel bottom connecting links comprising a top pivot pin hole at the first end of each bottom connecting link and a bottom pivot pin hole at the opposing second end of each bottom connecting link, the top pivot pin hole of each bottom connecting link pivotally connected to the bottom pivot pin hole of each respective posterior link by a first pivot axle.

9. The passive prosthetic knee of claim 8, wherein the rotatable latching member comprises:
a main body comprising a pivot pin hole provided through the main body, the pivot pin hole configured to receive the first pivot axle through the pivot pin hole, the first pivot axle pivotally coupling the main body with the bottom pivot pin hole of each respective posterior link;
a latching tongue protruded from the main body, the latching tongue configured to engage the spring lever responsive to the main body pivoting about the first pivot axle in a first direction; the latching tongue further configured to release the spring lever responsive to the main body pivoting about the first pivot axle in a second opposing direction; and
a first pair of parallel flanges extending from the main body, each flange of the first pair of parallel flanges comprising a respective first pivot pin hole, the first pivot pin hole of each flange configured to receive opposed end of a first pivot pin passing through a proximal pivot pin hole at the first end of the double-pivot rod.

10. The passive prosthetic knee of claim 9, wherein the bottom connecting plate further comprises a second pair of parallel flanges, each flange of the second pair of parallel flanges comprising a second pivot pin hole, the second pivot pin hole of each flange configured to receive opposed end of a second pivot pin passing through a distal pivot pin hole at the first end of the double-pivot rod.

11. The passive prosthetic knee of claim 10, wherein the spring lever further comprises a recessed portion at the proximal end of the spring lever, the recessed portion configured to receive the latching tongue within the recessed portion.

12. The passive prosthetic knee of claim 11, wherein the latching tongue moves into and engages with the recessed portion at the proximal end of the spring lever responsive to the main body pivoting about the first pivot axle in a first direction; the latching tongue moves out of and disengages the recessed portion at the proximal end of the spring lever responsive to the main body pivoting about the first pivot axle in a second opposing direction.

13. The passive prosthetic knee of claim 12, the double-pivot rod configured to urge the rotatable latching member to pivot about the first pivot axle in the first direction in response to the compression spring being longitudinally compressed between the middle connecting plate and the bottom connecting plate.

14. The passive prosthetic knee of claim 13, the double-pivot rod further configured to urge the rotatable latching member to pivot about the first pivot axle in the second direction in response to the compression spring being longitudinally decompressed between the middle connecting plate and the bottom connecting plate.

15. The passive prosthetic knee of claim 8, wherein each anterior link of the pair of parallel anterior links comprising a top pivot pin hole at the first end of each anterior link and a bottom pivot pin hole at the opposing second end of each anterior link, the top pivot pin hole of each anterior link pivotally connected to the anterior pivot pin hole of each respective top connecting link by a second pivot axle, the bottom pivot pin hole of each anterior link pivotally coupled to the bottom pivot pin hole of each respective bottom connecting link by a respective pivot pin of a pair of pivot pins.

16. A passive prosthetic knee, comprising:
a pair of parallel top connecting links;
a pair of parallel posterior links, a first end of each posterior link of the pair of parallel posterior links pivotally connected to a first end of each respective top connecting link;
a pair of parallel bottom connecting links, a first end of each bottom connecting link of the pair of parallel bottom connecting links pivotally connected to a second end of each respective posterior link; and
a pair of parallel anterior links, a first end of each anterior link of the pair of parallel anterior links pivotally connected to a second end of each respective top connecting link, a second end of each anterior link pivotally coupled to a second end of each respective bottom connecting link;
a stance spring selectively engaged with the pair of parallel posterior links;
a swing spring selectively engaged with the pair of parallel posterior links;
a compression spring longitudinally mounted between a middle connecting plate and a bottom connecting plate, each bottom connecting link of the pair of parallel bottom connecting links further attached to a respective side of the middle connecting plate;
a spring lever, a proximal end of the spring lever laterally coupled between the pair of parallel posterior links via middle pivot pin holes between the first end and the second end of each posterior link of the pair of parallel posterior links, an opposing distal end of the spring lever configured to contact the stance spring;
a rotatable latching member laterally coupled between the second ends of pair of parallel posterior links, the rotatable latching member configured to selectively latch onto the spring lever to prevent the pivotal movement of the spring lever with respect to the pair of parallel posterior links; and
a double-pivot rod, a first end of the double-pivot rod pivotally coupled to the bottom connecting plate, a second end of the double-pivot rod pivotally coupled to the rotatable latching member, the double-pivot rod configured to urge the rotatable latching member to latch onto the spring lever responsive to the compression spring being longitudinally compressed.

17. The passive prosthetic knee of claim 16, wherein the stance spring comprises:
a resilient elongated body longitudinally extended between a top end of the resilient elongated body and a bottom end of the resilient elongated body, the top end of the resilient elongated body in contact with the spring lever; and
a rigid elongated housing attached on the middle connecting plate, the resilient elongated body disposed within the rigid elongated housing, the bottom end of the resilient elongated body abutting against a base end of the rigid elongated housing, the top end of the resilient elongated body extended out of the rigid elongated housing.

18. The passive prosthetic knee of claim 17, wherein the spring lever comprises:
an extended part extended between the proximal end and the distal end of the spring lever, the extended part abutting against the top end of the resilient elongated body; and
a pair of parallel flanges at the proximal end of the spring lever, each flange of the pair of parallel flanges comprising a pivot pin hole, the pivot pin hole of each flange configured to receive opposed end of a pivot pin passing through the middle pivot pin holes of the pair of parallel posterior links, the spring lever pivotable relative to the pair of parallel posterior links at the proximal end of the spring lever about the pivot pin.

19. The passive prosthetic knee of claim 18, wherein the swing spring comprises a torsion coil spring mounted around the pivot pin, the torsion coil spring laterally disposed between the pair of parallel flanges.
